(19) United States
(12) Patent Application Publication
(10) Pub. No.: US 2014/0375470 A1
(43) Pub. Date: Dec. 25, 2014

(54) WEARABLE NETWORKED AND STANDALONE BIOMETRIC SENSOR SYSTEM TO RECORD AND TRANSMIT BIOMETRIC DATA FOR MULTIPLE APPLICATIONS

(71) Applicant: Chester Charles Malveaux, Baton Rouge, LA (US)

(72) Inventor: Chester Charles Malveaux, Baton Rouge, LA (US)

(21) Appl. No.: 13/923,335

(22) Filed: Jun. 20, 2013

(51) Int. Cl. A61B 5/00; H04Q 9/00; G01D 9/00

(52) U.S. Cl. CPC A61B 5/0015; G01D 9/00; H04Q 9/00; A61B 5/742; A61B 5/7275
USPC 340/870.01

(57) ABSTRACT

The invention provides for a wireless and data logging capable bio sensor system worn by individuals that transmits and stores biometric information gathered from multiple and diverse sensors on or near the individual's body. Through these sensors and its transmitters this device can convey and store vast amounts of information pertaining to an individual's physical condition and neurological state as well as the effects of physical activity on the individual's body. This data collection and sensory system could also be applied to biomechanical animal studies. Versions of this invention applied to the medical industry will allow medical specialists and patients to monitor health conditions in real time. This system will log historical biometric data to create an accurate mosaic of physical performances and conditions over a period of time. Logging of individual biometric data will create a unique and key biometric signature for each user.

### CROSS-REFERENCE TO RELATED APPLICATIONS

Not Applicable

### STATEMENT REGARDING FEDERALLY SPONSORED RESEARCH FOR DEVELOPMENT

Not Applicable

### REFERENCE TO SEQUENCE LISTING

Not Applicable

### BACKGROUND OF THE INVENTION

Sensors capable of recording health information and biometric data have been around for many years. Biometric sensor systems can allow for the recording of biometric data in the medical industry sensor data is crucial to the patient's standard of care and has been used for many years this type of data has also been applied to athletics and individual exercise. Sensor systems can make limited real time biometric data available to individuals. This invention can apply mathematical modeling and algorithms to the processing of specific sensor inputs in order to calculate physical stress and strain forces on the body of the wearer and improve the standard of care while also monitoring the progress of physical rehabilitation and physical exercise regimens, along with being used for security and emergency response functions greatly expanding the envelope of what biometric sensor systems can do.

### BRIEF SUMMARY OF THE INVENTION

This invention involves the creation of small light weight biometric sensor systems that store biometric data and can also transmit this data in real time to software applications so that this data can be analyzed, monitored, and cataloged. Large wired sensor systems are bulky and can impede a subjects range of motion, but by being small and self powered these sensors can be conveniently carried and or worn during the daily activities of the subject while not interfering with range of motion or physical capabilities. By logging and transmitting large amounts of biometric data while being worn or carried by the user these sensor systems will improve the monitoring of the progress of physical rehabilitation and exercise regimens while also offering patients and health care providers a vehicle which can be used to monitor real time health data in both clinical and nonclinical settings while also not disturbing the physical capabilities of the wearer. This data collection and sensory system could also be applied and effective for biomechanical animal studies such as for applications in the horse racing industry and domesticated animal and wildlife studies.

Often when a patient leaves the hospital it is not possible for that individual to be aware of their biometric information in real time and if they were aware of their health information in teal time it may be possible for them to seek help or obtain treatment for monitored conditions or even to simply take medications or preventative steps such as not over exerting themselves when sensors show that they are meeting or exceeding given parameters or if sensors detect as lack of a measured biometric response to treatment or exertion. Furthermore, with proper authorization this invention could make it possible for a physician or healthcare provider to be aware of a patient's condition in real time with software monitoring that could call for help in the event of a medical emergency. This system can also make health care providers and physical therapists aware of the effectiveness of physical therapy by logging physical therapy sessions and helping patients to be aware of the actual physical exertion performed and the effective biometric data generated by the exercise(s) or activities performed.

In the field of athletics this invention can make athletes, trainers, and individuals aware of the effectiveness of physical exercise and training regimens by providing real time data and data logging of activities and using mathematical models and algorithms to calculate the effects of activities and the physical forces exerted by the body of the athlete in real time and over historical periods. Using this biometric data exercise regimens can be improved and their effectiveness can be cataloged. In addition injuries can be avoided by using historical biomechanical data collected in order to create a predictive model that will help athletes, trainers, and individuals know when body stress and strain moments are exceeding recommended limits beyond which prior injuries have been observed in themselves or in others performing similar physical tasks or with similar physical conditions.

For security, first response, emergency preparedness and military applications individuals, teams, and commanders can be made aware of real time vital statistics and performance in the field of multiple groups as well as individuals during field operations of all types and during training exercises so as to optimize field performance. The speed and position of units can be tracked while also monitoring the physical condition of each unit member and maintaining situational awareness as to which unit members may need medical attention or assistance. Biometric and gps data will also allow full situational awareness as to the position and activities of individual unit members so that first responders and other emergency or security personnel can be more effective both collectively and individually. Real time streaming of this data as well as logging will allow for historical study of actions and allow adjustments that will make improvements to future responses to emergency situations as well as field exercises and general operations. The selective additional capability of allowing the biometric sensor units of individuals in the field to also display and log the position and biometric data and sensor information from other individuals in the field of operations will allow improved situational awareness of units and individual operators thus increasing the efficiency of field responses in theatres of operation. In addition each user will have a unique biometric signature recorded by the biometric sensor system which can be used in added security and other device features including control systems.

### BRIEF DESCRIPTION OF SEVERAL VIEWS OF THE DRAWING

FIG. 1 is a top view of an embodiment of the sensor and biometric data transmission system showing the outline of circuit boards containing the sensors, cpu, data storage, and transmitter. The cpu is visible at the center of the figure and is represented as a smaller rectangle held within a larger rectangle which is the main circuit board which holds the cpu along with data input and output pins. Next the heart rate sensor which can be mounted on various positions on the surface of the body of the wearer or within clothing is shown to the upper right of the main circuit board as a small triangle. To the right center of the main circuit board two small rectangles side by side represents the compass sensor and the accelerometer sensor. The accelerometer is the second rectangle to the right of the compass sensor. These sensors are to be placed on the body or worn in the clothing of the user. To the lower right of the main circuit board the gps sensor is shown as a small hexagon, this sensor can be worn or placed in various positions on the wearer limited only in that its actual position be advantageous for receiving gps satellite transmissions. To the upper left of the main circuit board are the radio modem and transmitters represented by an outward pointing arrow. To the center left of the main circuit board a circle represents a galvanic skin current sensor which is to be worn against the skin of the user for detection of changes in skin current which will help in determination of calories burned, exertion, perspiration, physical condition and other biometric data. To the lower left of the main circuit board a triangle represents spacing for additional optional biometric sensors including body temperature, moisture, contact pressure, shock, ambient, temperature, barometric pressure, water pressure, altitude, depth, stride, contact pressure between feet and surface of travel, and other sensors. At the bottom center of the main circuit board a medium sized hexagon represents neuroelectrical impulse sensors.

FIG. 2 is a view showing an outline of the body where sensors and displays for the biometric sensor system could be placed. Small hexagons represent biometric sensor locations, and rectangles represent locations for sensor displays including eye area displays near the head area of the figure and wrist mounted displays depicted at the ends of the left and right arms of the figure. At the upper section of the diagram the top most hexagon represents neuroelectrical impulse sensors and at the lower section of the drawing the lower most hexagons represent pressure and stride sensors to detect movement and pressure related sensor inputs.

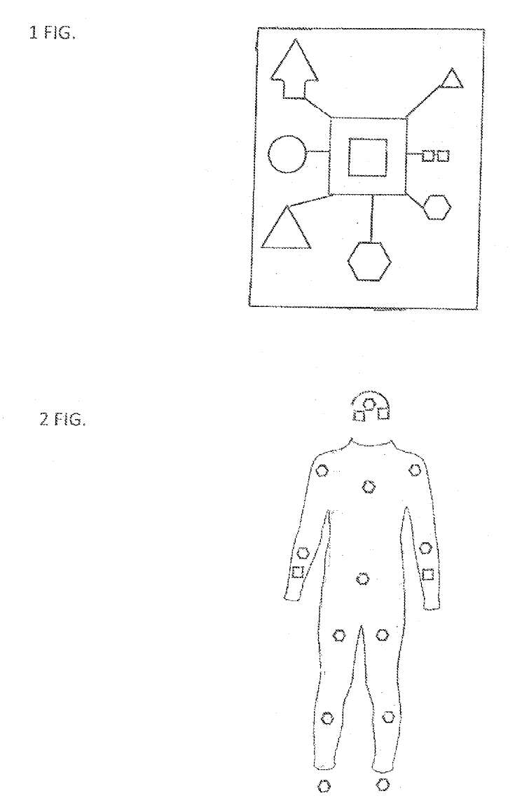

### DETAILED DESCRIPTION OF THE INVENTION

The present invention relates generally to a biometric sensor system and networked biometric sensors. More particularly, the present invention relates to light weight networked and individual biometric sensors which can be worn by users and provide a myriad of biometric data which can then be used by wearers and or concerned parties to monitor physical condition and physical performance while also cataloging and recording the same. These sensors will allow the wearers to log biometric data and also to transmit this biometric data to authorized parties for observation, cataloging, and processing of data in order to assist with various individual needs ranging from optimized health management to physical conditioning and emergency preparedness along with other needs which can be met by cataloging, recording, and monitoring real time biometric data.

By wearing the sensors individuals can catalog the amount of physical activity which they have performed and also monitor the effects of this activity on their body. Algorithms will be used to quantify the physical effects of exercise on individuals. Through applications allowing user interface and spreadsheet software the system will allow users to monitor progress and observe physical condition over time while also being able to analyze historical biometric data and performance. By observing historical data injuries can be avoided in that stresses and strains which caused previous injuries will be cataloged and when activities approach levels which historical data shows caused injuries in the user or others alarms can be set to warn the user and or authorized observers.

Individuals who have medical conditions will be able to monitor their physical condition and also allow this data to be shared with healthcare personal via wireless link or through secured interact connection while also logging biometric data for detailed discussion during future consultations. Additionally applications capable of running on smart phones and tablets will allow individuals and any authorized parties to observe biometric data in real time.

The best mode for carrying out the invention is provided by existing microcontroller and sensor technology. Fabrication of light weight and water proof housings for sensors and transmitters is readily available. Current wireless modern technology and data transmission security features are sufficient to be applied to the applications of this device. Computer aided design and 3D printing technology makes this device buildable.

Biometric sensors have been around for some time, but wireless networked biometric sensors capable cit cataloging and storing a unique biometric signature for each user and also communicating location and condition for numerous users while combining and cataloging the input of numerous networked biometric sensors and sensor systems into a linked network and using mathematical formulas and algorithms to calculate body stress and strain while taking into account the mass of the individual is an innovative design that will be of great benefit to users along with security and emergency response personnel. In addition the onboard processing capability of these biometric sensor systems will allow them to provide expanded functionality by using biometric data including neuroelectrical impulse data to allow the biometric sensor system to act as an access key and a control system for devices and machinery while also securing itself and preventing unauthorized access to the biometric sensor system and network.

What is claimed is:

1. A biometric sensor, recording, and wireless biometric data transmission system that can record and transmit biometric data to any graphical user interface, spreadsheet software, and smart phone, tablet, watch, heads up display system, any worn visual display system, and other device with application software to allow for real time observation as well as historical recording and analysis of biometric data with onboard processing and biometric data storage capability.

2. A biometric sensor system of claim 1 wherein mathematical formulas and algorithms allow biomechanical stress and strain at given points on an individual's body to be recorded and cataloged so as to make individuals and authorized parties aware of the potential for injury and also the degree to which the individual has exercised that section of their body.

3. A biometric sensor system of claim 1 wherein gps, 3-axis accelerometer, heart rate, galvanic skin response, 3-axis compass position, body temperature, and additional biometric sensors are combined to give an accurate understanding of an individual's physical performance and physical state in real time.

4. A biometric sensor system of claim 3 wherein the capability of transmitting biometric data in real time and recording these measurements for future observation and analysis is included as part of the system.

5. A biometric sensor system of claim 1 wherein real-time biometric data is transmitted to web based, pc based, and application based software for processing and observation over internet and intranet based user interface devices including but not limited to smart phones, tablets, personal computers, terminals, and wireless monitoring devices belonging to the wearer of the biometric sensor and or belonging to authorized parties and or groups of device users.

6. A biometric sensor system of claim 1 wherein real-time biometric sensor data from multiple sensors is networked forming groups of biometric sensors all working at the same time and communicating with each other.

7. A biometric sensor system and user interface of claim 6 wherein multiple biometric wireless data streams and historical biometric data from multiple sensors can be observed by authorized users of networked sensors and by other authorized parties for real time situational awareness, real time awareness of the physical condition and physical location.

8. A biometric sensor system of claim 1 wherein biometric data is displayed in real time and also stored for observation and optimization of physical performance in both real time and based on observed historical performance and position data.

9. A biometric sensor system of claim 1 which is water proof and integrated into clothing which provides biometric data to the wearer and or authorized parties and other networked biometric sensors at diverse locations through wireless and wired interfaces.

10. A biometric Sensor system of claim 6 wherein wireless and wired biometric sensor placed at various locations on the head, body, helmet, apparel, footwear, and other wearable or attached gear and or clothing of the user which have sensors embedded and can transmit and receive data shared between biometric sensors from diverse body locations ranging from pants to boots, gloves, shirts, hats, eyewear, shoes and other segments of clothing via wireless and or wired data connections between sensors and then displayed on or in worn graphical user interfaces and heads up display systems worn or affixed to outside displays, and transmitted to web based, pc based, and application based soft-ware for processing and observation over online and intranet based user interface devices including but not limited to smart phones, tablets, personal computers, terminals, and wireless monitoring devices to include video display and heads up display devices worn by the wearer of the biometric sensor and or authorized parties.

11. A biometric sensor system of claim 1 wherein groups of networked addressable biometric sensors receive and retransmit the radio frequency signal of biometric sensors within range to other networked biometric sensors thereby creating an extended range sensor network that can stretch out to any networked sensors in range even if a networked biometric sensor system is out of range of the networked biometric sensor system geographically farthest away from its location provided that other networked biometric sensors are in range to carry its addressable signal to that farthest away biometric sensor system.

12. A biometric sensor system which uses fixed point radio frequency broadcasts from multiple antennas or radio frequency emitters to triangulate and maintain the position of all biometric sensors so as to augment gps positioning and work in areas where gps positioning is not available.

13. A biometric sensor system fitted with receivers and transmitters allowing it to receive and transmit radio frequency emissions in order to triangulate its position via radio telemetry and report its position using an onboard computer processor while also broadcasting its location to other networked biometric sensors within its range which are capable of also performing these same functions with all biometric sensors also able to repeat the transmission signals and locations of biometric sensors in within the range of the receiving biometric transmitter.

14. A biometric sensor system of claim 1 with preset sensor readings which if reached trigger an alert response to warn the user and authorized parties that preset physical conditions have been registered by the biometric sensors worn by the user.

15. A biometric sensor system of claim 1 wherein biometric sensor systems can be removed by users and linked to a base station for recharging and downloading of stored biometric, movement logs, and other sensor data.

16. A biometric sensor system of claim 1 wherein by recording the specific biometric data of the user the device will be capable of creating a biometric identifier for each individual user and this identifier can be used as a lock and key system to open or restrict the functionality of this device and also to open and restrict the functionality of weapons systems, electronics, doors, locks, machinery or other equipment to which it is linked thereby becoming biometric lock and key mechanism and also ensuring that the device and equipment is not misused and or that its data and network system is not accessed by those without authorization.

17. A biometric sensor system of claim 1 wherein encryption and special signal processing is used to ensure that data and network information security is not compromised or accessible to unauthorized personnel.

18. A biometric sensor system of claim 1 wherein additional biometric sensors can be used to register alpha, beta, theta, delta, and gamma brainwaves along with other neuro-electrical impulses from the wearer in order to give biofeedback data that can be used to help the wearer and authorized personnel access the neurological state of the biometric sensor system user and also to monitor and control neurologic state and other biofeedback related neurological or neuromuscular states as for example may be needed for forms of physical therapy or physical conditioning and training.

19. A biometric sensor system of claim 17 wherein registered neuro-electrical impulses can be amplified and converted into pulse width modulated control signals by the biometric sensor system to activate and run control systems and training simulations allowing the user to master the control of machinery and other devices through the thoughtful modulation of neuro-electrical impulses as observed outputs from the biometric sensor system which is also amplifying these neuro-electrical impulses and converting them into control signals for devices, equipment, and machinery.
